(19) United States
(12) Patent Application Publication
(10) Pub. No.: US 2014/0221864 A1
(43) Pub. Date: Aug. 7, 2014

(54) ELECTRIC IMPEDANCE TOMOGRAPHY DEVICE AND METHOD

(71) Applicant: Drager Medical GmbH, Lubeck (DE)

(72) Inventor: Yvo GÄRBER, Breitenfelde (DE)

(21) Appl. No.: 13/943,271

(22) Filed: Jul. 16, 2013

(30) Foreign Application Priority Data
Feb. 5, 2013 (DE) 10 2013 201 806.1

(51) Int. Cl. A61B 5/053

(52) U.S. Cl. CPC A61B 5/0536
USPC 600/533

(57) ABSTRACT

An EIT device with a plurality of electrodes, which can be arranged about the chest of a patient, with a control and analyzing unit for feeding electrode pairs of a set of electrodes to record a voltage or current signal as a measured signal with electrode pairs acting consecutively as the feeding electrode pair to provide a matrix of image elements. A time series of the impedance change from the sequence of reconstructed matrices over at least one breath is obtained and compared to a determined time series of the mean impedance change or a time series of a measured respiration volume, by calculating for each image element a scalar value as an indicator of a deviation. The control and analyzing unit assesses and marks the corresponding image element as being non-ventilated if the indicator of the deviation meets a preset threshold value criterion.

### CROSS REFERENCE TO RELATED APPLICATIONS

This application claims the benefit of priority under 35 U.S.C. §119 of German Patent Application DE 10 2013 201 806.1 filed Feb. 5, 2013, the entire contents of which are incorporated herein by reference.

### FIELD OF THE INVENTION

The present invention pertains to an electric impedance tomography device with a plurality of electrodes that can be arranged about the chest of a patient, with a control and analyzing unit, which is set up by programming (configured) to supply an electrode pair as a feeding electrode pair with an alternating current or with an alternating voltage, to record a voltage signal or current signal as a measured signal from each electrode pair of all other electrode pairs, and to let each electrode pair of a plurality of electrode pairs act consecutively as the feeding electrode pair in order to reconstruct from the measured signals with a reconstruction algorithm a matrix of image elements, which represents the distribution of the impedance changes in the electrode plane and to repeatedly record measured signals over time and to reconstruct matrices.

### BACKGROUND OF THE INVENTION

Such an electric impedance tomography device (EIT device) is known, for example, from EP 1 000 580 A1, which is used to record an “electric impedance tomogram” of a chest cross section of a patient.

Electric impedance tomography is a method for the reconstruction of impedance distributions, more precisely, of impedance changes relative to a reference distribution, in electrically conductive bodies. A plurality of electrodes are arranged for this purpose on the surface of the body to be examined. A ring-shaped, equidistant array of 16 electrodes, which can be laid about the chest of a patient with a belt, is used in typical cases. The control and analyzing unit also has analog electric circuits for signal amplification and for feeding alternating current and electronic circuits for digitizing and preprocessing the voltage signals as well as a digital signal processor for controlling the device and for processing the recorded data to reconstruct the impedance distribution. The control and analyzing unit ensures that always one pair (preferably) of adjacent electrodes is supplied consecutively with an electric alternating voltage (e.g., 5 mA at 50 kHz) and that the electric voltages on the remaining electrodes are detected by the control and analyzing unit (it is, in principle, also possible, conversely, to feed an alternating voltage to one electrode pair and to measure the alternating currents over the other electrode pairs). The impedance distribution, more precisely, the impedance change distribution relative to a reference distribution, can be reconstructed with algorithms from all the measured signals during the consecutive current feeds, during which the feeding electrode pair migrates step by step around the electrode ring. The prior-art algorithms furnish as the reconstruction result a matrix of 32×32 image elements, and the matrix for each image element contains the reconstructed impedance change for that image element. A plurality of such matrices are recorded during each breath at preset time intervals. These are displayed consecutively on a display, as a result of which the changes in the impedance distribution over time are made visible practically as a film.

Electric impedance tomography of the chest for measuring the regional lung ventilation has been increasingly used in research-focused intensive care. Theoretical models and experimental comparisons of EIT with CT images of the chest show a nearly complete proportionality of the air content in the lung tissue to the impedance thereof. The breaths are resolved in space with about 20% of the chest diameter and in time typically with about 20 to about 40 matrices per second, which makes bedside monitoring of the regional lung ventilation possible. The matrices are occasionally also called images of the impedance distribution (with 32×32=1024 image elements) or frames.

The terms time series of impedance change values and impedance change curves will hereinafter be used with the same meaning, even though a time series consisting of discrete dots is not a curve in the strict sense of the word. The time series are also represented in the form of curves as functions of time for reasons of representation in views as well.

A mean impedance change for each matrix is obtained by integration and standardization or averaging over all image elements of the matrix. A time series of the mean impedance change can thus be derived from the sequence of matrices recorded consecutively. This represents the global impedance change, contrary to the local impedance change of the individual image elements. Based on the nearly complete proportionality between tidal air volume change and impedance change, the mean impedance change time series or impedance change curve is closely correlated with the measured volume curve of a respirator respirating the patient, i.e., the impedance change curve agrees nearly completely, except for a standardization, with the volume curve of respiration. This is shown in FIG. 1, in which the volume curve 2 of the respirator is compared with the impedance change curve 1, which is standardized to the same integral. No deviation of the volume curve 2 from the mean impedance change curve 1 is recognizable over one breath.

If the lung function is homogeneous in space and over time, it follows from this that the correlation of the time series of the impedance changes of the individual image elements of the matrices with the mean impedance change curve and hence also with a possibly measured volume curve of the respirator is very close. This is shown in FIG. 2, in which the mean impedance change curve is shown over one breath at the top and the impedance change curves of four individual image elements 1, 2, 3 and 4, whose locations are indicated in the upper EIT image, are shown under it. The examples in FIG. 2 show that the correlations c marked with reference number 6 with the mean impedance change curve equal 100% for all four image elements.

Weaker correlations are to be expected in case of inhomogeneous ventilation over time, because the air flows at different velocities into different areas of the lungs in this case, but it remains valid in this case as well that, aside from redistributions, the impedance essentially increases with increasing volume. The mean impedance change curves of four image elements from a ventilated COPD lung (Chronic Obstructive Pulmonary Disease) that is highly inhomogeneous over time are shown again in the top part of FIG. 3. The correlations indicated for the four image elements are weaker here and are between 78% and 98%.

However, there also are cases in which the local characteristic over time does deviate from the mean or global characteristic; the impedance may even decrease with increasing volume in the extreme case. This is shown in FIG. 4, in which the mean impedance change curve is shown again at the top and the impedance change curves of four individual image elements are shown under it. The image elements 3 and 4 from the dorsal area of the lung show correlations of −95% and −80%, i.e., anticorrelations. This could be caused, on the one hand, by a reconstruction artifact, which is called overshoot. However, this could also be due to actual physiological effects, whose causes have not yet been fully elucidated. Displacements of tissues due to motion of the diaphragm into the electrode planes if the electrodes are placed too deep, or even displacements of fluids in case of massive atelectases or pleural effusions are assumed here. Regardless of the specific causes, it would be desirable to make it possible to recognize and mark image elements whose impedance change curves deviate greatly from the mean impedance change curve.

### SUMMARY OF THE INVENTION

An object of the present invention is to design an electric impedance tomography device such that image elements whose local impedance change over time deviates significantly from the mean impedance change curve can be recognized and marked.

According to the invention, an EIT device is provided with a plurality of electrodes, which can be arranged about the chest of a patient and with a control and analyzing unit. The control and analyzing unit is configured (set up by programming) to supply an electrode pair as a feeding electrode pair with an alternating current or with an alternating voltage, to record a voltage signal or current signal as a measured signal from each electrode pair of all other electrode pairs and to let each electrode pair of the plurality of electrode pairs act consecutively as the feeding electrode pair in order to reconstruct from the measured signals with a reconstruction algorithm a matrix of image elements, which represents the distribution of the impedance changes in the electrode plane, and to repeatedly record measured signals over time and to reconstruct matrices. The control and analyzing unit is configured, furthermore, to obtain a time series of the impedance change from the sequence of reconstructed matrices over at least one breath, to determine a time series of the mean impedance change or a time series of a measured respiration volume and to compare for each image element the corresponding time series of the impedance change with the time series of the mean impedance change or with the time series of the measured respiration volume by calculating for each image element a scalar value as an indicator of the deviation of the time series of the impedance change of the image element from the time series of the mean impedance change or from the time series of the measured respiration volume. The control and analyzing unit assesses and marks the corresponding image element as being non-ventilated if the indicator of the deviation meets a preset threshold value criterion.

According to another aspect of the invention, a method is provided for recording a sequence of EIT images of a cross-sectional plane of the chest of a patient. The method comprises providing a control and analyzing unit connected to a plurality of electrodes, arranging the plurality of electrodes on the circumference of the chest of the patient, wherein one electrode pair as a feeding electrode pair is supplied with an alternating current or an alternating voltage, a voltage signal or current signal is recorded as a measured signal from each electrode pair of all other electrode pairs, and each electrode pair of the plurality of electrode pairs is operated consecutively as the feeding electrode pair. The method uses the control and analyzing unit for reconstructing a matrix of image elements, which represents the distribution of the impedance changes in an electrode plane, from all the measured signals with a reconstruction algorithm, reconstructing matrices of the impedance change repeatedly over time, obtaining a time series of the impedance change of the image element from the sequence of the matrices recorded over at least one breath for each image element and determining a time series of the mean impedance change or a time series of a measured respiration volume is determined at the same times as the time series of the image elements. The control and analyzing unit compares a corresponding time series of the impedance change, for each image element, with the time series of the mean impedance change or with the time series of the measured respiration volume by calculating for each image element a scalar value as an indicator of the deviation of the time series of the impedance change of the image element from the time series of the mean impedance change or of the time series of the measured respiration volume. The control and analyzing unit assesses a corresponding image element as being non-ventilated and marks the corresponding image element as such if the indicator of the deviation meets a preset threshold value criterion.

Provisions are made according to the present invention for the control and analyzing unit to be set up to obtain a time series of the impedance change in the given image element from the time series of the matrices recorded, preferably over at least one breath, for each image element. Furthermore, a time series of the mean impedance change (each averaged over one matrix) is determined from the sequence of matrices, or a time series of a measured respiration volume is determined; the time series of the measured respiration volume would then, of course, be set up such that it pertains to the same times as the times at which the matrices of the impedance changes are recorded. Furthermore, the control and analyzing unit is set up to compare, for each image element, the corresponding time series of the impedance change with the time series of the mean impedance change or with the time series of the measured respiration volume by calculating for each image element a scaled value as an indicator of the deviation of the corresponding time series from the time series of the mean impedance change or of the time series of the measured respiration volume. If the indicator of the deviation of the time series from one another meets a preset threshold value criterion, the corresponding image element is assessed by the control and analyzing unit as non-ventilated and is marked correspondingly.

One advantage of the present invention is that an unusual characteristic may suddenly appear when the ventilation parameters are changed during artificial respiration, for example, a PEEP (positive end-expiratory pressure) change, and that it becomes visible according to the present invention in a short time where non-ventilated areas are present and what possible percentage of the lung is not ventilated. The physician can then either adjust the PEEP in a specific manner until the unusual characteristic disappears, and/or attempt to specifically find the morphological cause of the changed pattern by other imaging methods, such as computed tomography or magnetic resonance tomography.

Another advantage of the present invention is that the marked non-ventilated image elements can be excluded from further analyses of the local ventilation, e.g., RVD (Regional Ventilation Delay) (T. Muders et al., Regional ventilation delay index: Detection of tidal recruitment using electric impedance tomography, Vincent J. L., Editor, Yearbook of intensive care and emergency medicine), and ITV (Intratidal Variation—K. Lowhagen et al., Regional intratidal gas distribution in acute lung injury and acute respiratory distress syndrome—Assessed by electric impedance tomography, Minerva Anestesiol., 76 (2010, 1024) and the quality of further analyses of local ventilation can thus be improved. Erroneous results, which appear otherwise because the preconditions for such analysis (increase in impedance during air supply and vice versa) are not met for these non-ventilated image elements, can thus be avoided.

The correlation coefficient between the time series of the impedance change and the time series of the mean impedance change or the time series of the measured respiration volume can be calculated as the indicator of the deviation for each image element. Whether the correlation coefficient is below a preset value is then checked as the threshold value criterion. A value of 1 of the correlation coefficient means that the two time series show no deviations from one another, and a value of 0 means that there is no relationship between them, and a value of −1 means that the time series behave exactly oppositely to one another. If, for example, the time series of the impedance change of an image element k is designated by zₖ(tᵢ), I=1, . . . , m and the time series of the mean impedance change (the subscript glo denotes “global”) is designated by z_{glo}(tᵢ), I=1, . . . , m, the correlation coefficient for the image element k can be calculated according to the following formula:

$${c}_{k} = \frac{\frac{1}{m} {\sum }_{i = 1}^{m} ( {z}_{k} ( {t}_{i} ) - {\overline{z}}_{k} ) \cdot ( {z}_{glo} ( {t}_{i} ) - {\overline{z}}_{glo} )}{\sqrt{\frac{1}{m} {\sum }_{i = 1}^{m} {( {z}_{k} ( {t}_{i} ) - {\overline{z}}_{k} )}^{2}} \cdot \sqrt{\frac{1}{m} {\sum }_{i = 1}^{m} {( {z}_{glo} ( {t}_{i} ) - {\overline{z}}_{glo} )}^{2}}}$$

in which the values provided with a dash represent the mean values of the time series over the times tᵢ, I=1, . . . , m. The correlation coefficients are in a value range of [−1, 1]. The correlation in percentage, which assumes values in the range of [−100%, 100%], is obtained by multiplication by 100. The correlation is usually stated in connection with the exemplary embodiments.

As an alternative, the cross correlation function of the time series of the impedance change and of the time series of the mean impedance change or of the time series of the measured respiration volume can be calculated for each image element. The maximum of the cross correlation function is then determined as the indicator of the deviation, and the possibility that the maximum exceeds a preset value is checked as the threshold value criterion.

As an alternative, the indicator of the deviation can be calculated by considering the standardized time series of the impedance change of the image element to be a vector and considering the standardized time series of the mean impedance change or the standardized time series of the measured respiration volume to be a vector. The standardization of the time series is performed by division by the integral value thereof or the maximum thereof or generally by a norm of the vector space of the time series (1-norm, 2-norm, etc.). (A norm is generally an imaging of each vector of the vector space to a real number, which meets the following conditions: The norm of the zero vector is 0, the norm of vector α19 \vec{V} (α being a real number) equals multiplied by the norm of \vec{V}, and the triangle inequality applies. The value of the difference of said vectors is calculated as an indicator of the deviation. It is checked as the threshold value criterion in this case whether the norm of the difference exceeds a preset threshold value. If the value of the difference of the vectors exceeds a preset threshold value, this means that these deviate from each other considerably at least at certain times of the time series.

In an alternative embodiment, the standardized time series of the impedance change of the image element is again considered to be a vector and the standardized time series of the mean impedance change or the standardized time series of the measured respiration volume is considered to be a vector and the scalar product of the two vectors mentioned is formed as an indicator of the agreement. It is then checked as a threshold value criterion whether the value of the scalar product is below a preset threshold value. If the value of the scalar product is low, this means that the two vectors are positioned nearly at right angles to each other.

The indicator of the deviation for each image element is calculated in another embodiment by considering the standardized time series of the impedance change to be a vector and by considering the standardized time series of the mean impedance change or the standardized time series of the measured respiration volume to be a vector and calculating the standard of the sum of the said vectors as an indicator of the deviation, and it is checked as a threshold value criterion whether the standard of the sum is below a preset threshold value. If the sum is small, this means that the time series do not complement each other constructively but behave rather opposed to each other, which corresponds to a negative correlation.

The control and analyzing unit can be set up in an advantageous embodiment to display non-ventilated image elements assessed as being non-ventilated in an EIT image by a preset shade.

In another embodiment, the control and analyzing unit can be set up to determine the sum of the areas of the image elements assessed as being non-ventilated and to display these graphically or alphanumerically or to display the ratio thereof to the entire cross-sectional area of the lungs graphically or alphanumerically with an EIT image.

The control and analyzing unit can be set up to perform the reconstruction of the matrices from the measured signals in real time and to determine the indicator of the deviation for each image element in real time and to check the threshold value criterion in real time and to mark image elements assessed as being non-ventilated in a current EIT image.

As an alternative, the control and analyzing unit can be set up to store the measured signals and to perform the reconstruction of the matrices and the formation of the time series of the impedance change of the image elements and of the time series of the mean impedance changed or of the time series from the stored measured respiration volume in a subsequent analysis and to determine for each image element the indicator of the deviation of the time series of the image element from the time series of the mean impedance change or from the time series of the measured respiration volume and to check for the threshold value criterion and to assess image elements that meet a preset threshold value criterion as being non-ventilated.

Furthermore, the present invention provides for a method for recording a sequence of EIT images of a cross-sectional plane of the chest of a patient, on the circumference of which a plurality of electrodes are arranged, wherein an electrode pair as a feeding electrode pair is supplied in the method with an alternating current or an alternating voltage; a voltage signal or current signal is recorded as a measured signal by each electrode pair from all other electrode pairs, and each electrode pair of a plurality of electrode pairs is operated consecutively as the feeding electrode pair; a matrix of image elements, which represents the distribution of the impedance changes in the electrode planes, is reconstructed from all the measured signals with a reconstruction algorithm, and matrices of the impedance change are reconstructed repeatedly over time (preferably over at least one breath), wherein a time series of the impedance change of the image element is obtained from the sequence of the recorded matrices for each image element, a time series of the mean impedance change or a time series of a measured respiration volume is determined at the same times as the time series of the image elements, and the corresponding time series of the impedance change is compared for each image element with the time series of the mean impedance change or with the time series of the measured respiration volume by calculating a scaled value for each time series as an indicator of the deviation of the time series of the impedance change of the image element from the time series of the mean impedance change or of the time series of the measured respiration volume. If the indicator of the deviation meets a preset threshold value criterion, the corresponding image element is assessed as being non-ventilated and is marked as such.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a respiration volume curve and a mean impedance change curve over one breath in comparison;

FIG. 2 is, in the top parts, a mean impedance change curve over one breath and, under it, four impedance change curves for four selected image elements;

FIG. 3 is, in the top parts, a mean impedance change curve over one breath and, under it, four impedance change curves for four selected image elements;

FIG. 4 is, in the top parts, a mean impedance change curve over one breath and, under it, four impedance change curves for four selected image elements; and

FIG. 5 is a schematic view showing an EIT device with electrodes and control and analyzing unit.

### DESCRIPTION OF THE PREFERRED EMBODIMENTS

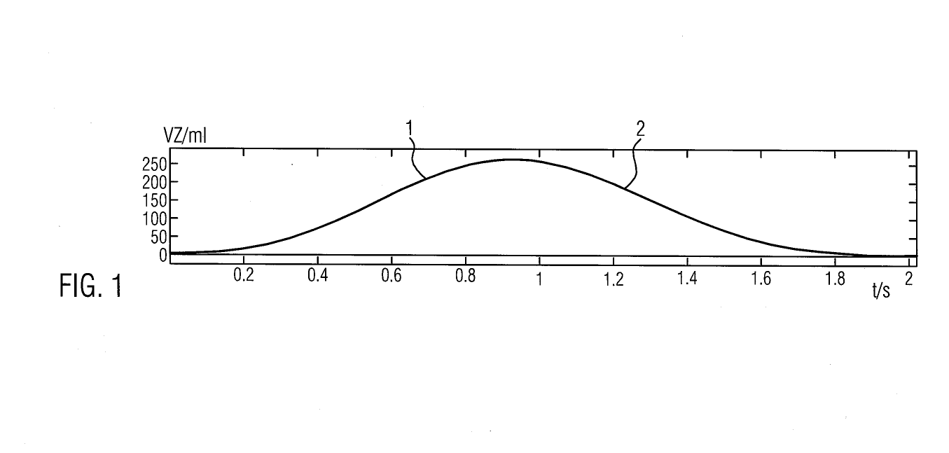

Referring to the drawings in particular, FIG. 1 shows the mean impedance change curve 1 and a respiration volume curve 2 measured by the respirator as a function of time over one inspiration and subsequent expiration, wherein the two curves agree with one another so much that they are not resolved in FIG. 1.

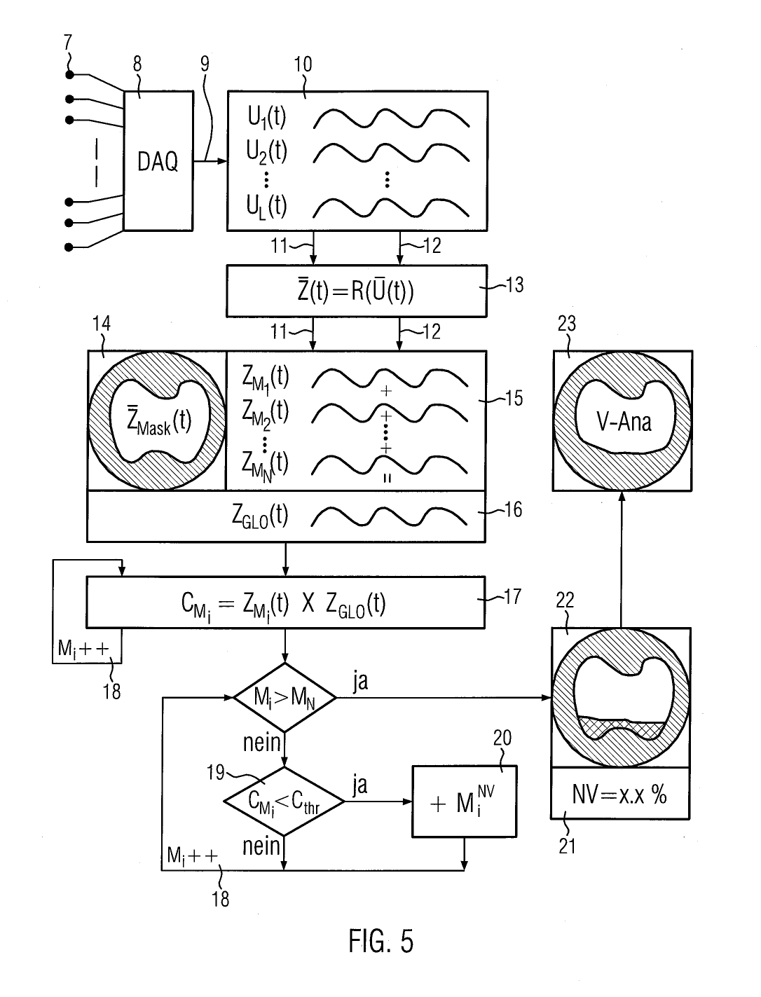

The design and mode of operation of the EIT device shown schematically in FIG. 5 will first be described now. The EIT device has electrodes 7, which can be arranged in a ring-shaped pattern about the chest of a patient. The remaining components shown are part of the control and analyzing unit, except for the display means 22 and 23, and different functions of the control and analyzing unit are shown here in separate modules. However, this does not mean that these modules have to be physically separate units. The different functions of the control and analyzing unit can rather be embodied in a data processor, wherein the different modules shown in FIG. 5 are in this case embodied in different program units.

In typical cases, EIT devices have 16 electrodes. A data acquisition unit 8 of the EIT device ensures current feed via an electrode pair and the recording of the measured voltages between the other electrode pairs. Alternating current is then fed via a next electrode pair and the measured voltages of all other electrode pairs are recorded, etc., until each electrode pair has functioned once as a feeding electrode pair. Thus, a recording of 208 measured voltages is obtained for a device with 16 electrodes; these 208 measured voltages recorded are also called frames. Typical EIT devices operate with frame rates between 10 Hz and 50 Hz. The 208 measured voltages are sent to the storage unit 10 of the EIT device via a bus system 9.

During the real-time processing of the data, a reconstruction module 13 processes the measured voltages and reconstructs from them a matrix of image elements (typically 32×32=1024 image elements), which represent the local distribution of the impedance change. The measured voltages are stored in the alternative data processing at a later time. This is indicated schematically by the further connection line 12, which shall mean that the measured voltages are stored at first and are sent to the further processing steps later.

In the time series module 15 for the time series of the image elements, the image elements are added up and standardized in order to obtain a time series of the mean impedance change z_{glo}(t), which represents the global impedance change characteristic and is made available in a module 16 for the mean impedance change.

It is not necessary to take all image elements into account, because the cross-sectional image of a torso, which is represented in a square grid of image elements, leaves areas behind, in corners, which do not belong to the torso and therefore also should not be included in the analysis. This is indicated in FIG. 5 by the white corners 14 around a schematic representation of a tomogram through a torso. However, image elements that are located outside the lung area may be present within the reconstructed area as well. For example, there are strong muscle strands in the dorsal region in animal experiments carried out on pigs. There is a marginal fat layer in obese patients. A mask prepared with other means and methods, which blanks out the outer area that is of no interest and selects only the information-carrying lung image elements, is therefore used. In FIG. 5, the selected image elements are designated in the time series module 15 by M₁(t), . . . , M_{N}(t), which designate the subscripts of the time series of the impedance change of the analyzed image element.

The time series for the impedance change z_{Mi}(t) is linked in the correlation module 17, 18 with the time series of the mean impedance change z_{glo}(t) for each image element Mᵢ in order to form a correlation coefficient c_{Mi} for each image element (it should be noted that the time series are designated in FIG. 5 simply as functions of the time t, but this shall represent a simplification of the representation only, because what is actually meant is time series at discrete times). The link of the time series, which is shown in correlation module 17, 18, is shown as a formula as a link only symbolically; the actual formulation of the link is represented, for example, in the formula shown explicitly above for the correlation coefficient.

After forming the correlation coefficients in correlation module 17, a loop is performed again over all selected image elements. This is schematically shown in the bottom part of FIG. 5. A loop is performed at first over all image elements M₁, . . . , M_{N} and a polling is performed to determine if the image element being considered is still smaller than the maximum image element M_{N} of the image elements selected through the mask. If the subscript of the image element is still lower, a polling is performed to determine whether the correlation coefficient c_{M1} is lower than the threshold value cₜₕᵣ. If yes, image element Mᵢ is marked as non-ventilated image element in the marking module 20. As soon as all image elements selected through the mask have been processed, the EIT image is displayed on a display means 22, and the image elements assessed as being non-ventilated are represented with a special marking on the display means. Furthermore, a display module 21, which shows the percentage of non-ventilated image elements, is provided for the non-ventilated component.

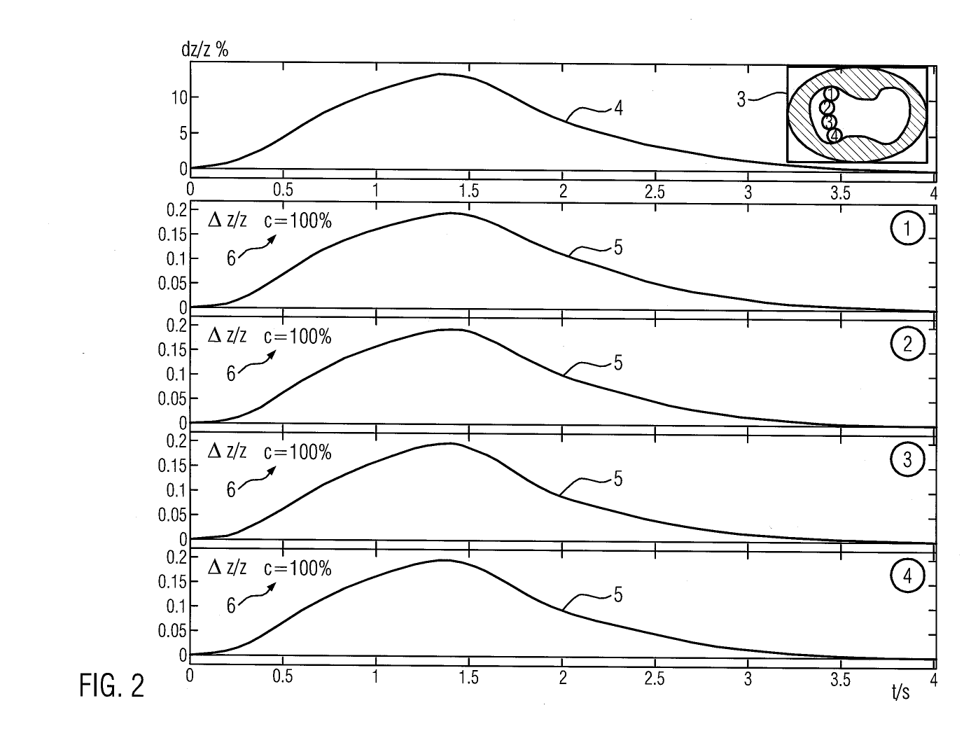

Examples of the mode of operation of the present invention are shown in FIGS. 2 through 4, in which the correlation between the time series is selected as the indicator of the deviation. The mean impedance curve (or the time series of the averaged impedance change) is always shown in the upper graphs in the figures. The time series or curves of the impedance change for individual, selected image elements 1 through 4 are shown in the four graphs under it.

The ventilation of the lung is rather homogeneous in space and time in the first case in FIG. 2. The correlations with the mean impedance change curve1 equals, after rounding, 100% for all selected image elements 1, 2, 3 and 4. All image elements within the lung area of the cross-sectional image are ventilated.

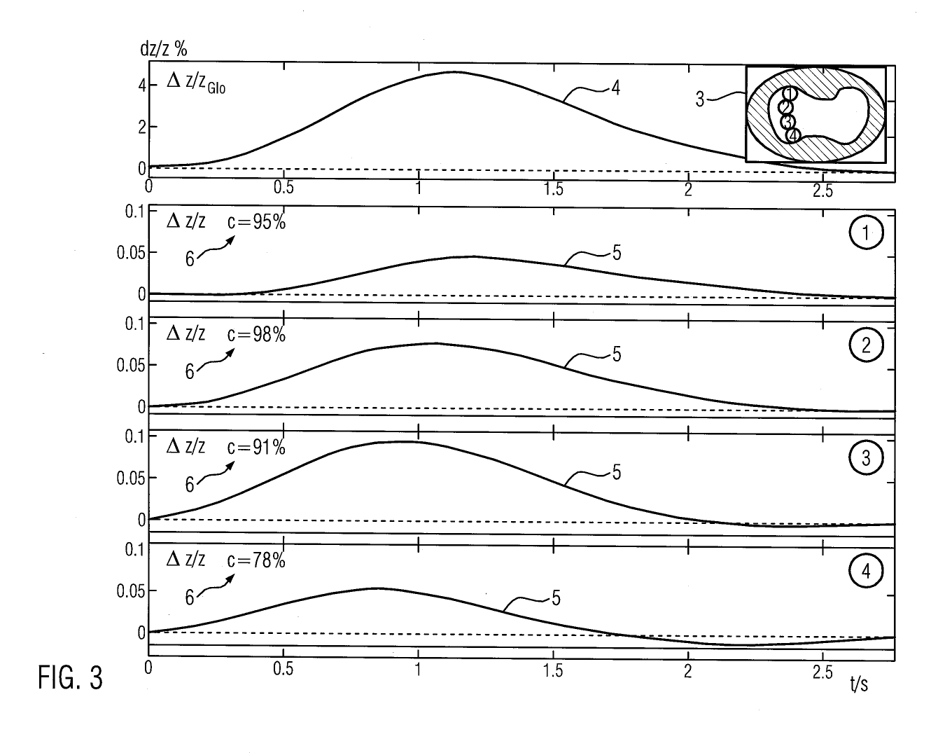

The lung in FIG. 3 is a COPD lung, which is ventilated with great offset in time because of different regional resistivities and elasticities. The dorsal regions are ventilated earlier than the ventral ones, but the pattern of the local image element curves 5 still agrees essentially with the averaged impedance curve 4, so that the correlations are still always rather close at nearly 80% to 100%. The cross correlation curves, not shown, all have their maxima at 100%.

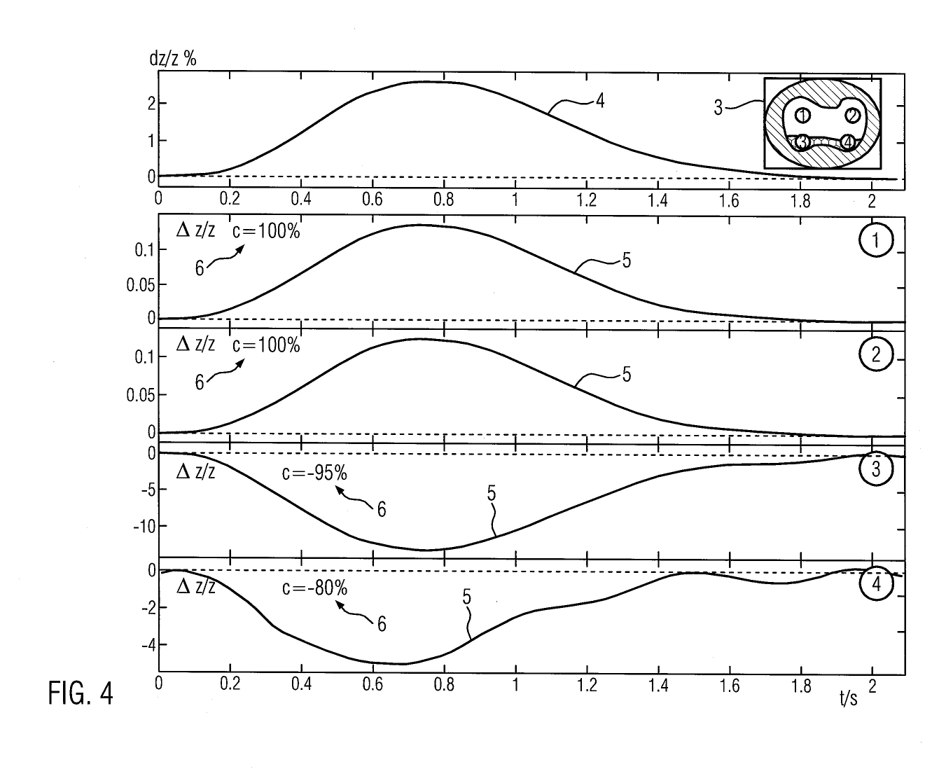

FIG. 4 shows an animal experiment with a lung artificially damaged with hydrochloric acid. The curves or time series of the image elements in the central right and left lung area with the numbers 1 and 2 show excellent correlation with the mean impedance change curve with correlations of 100%, whereas the impedance change curves of the image elements 3 and 4 in the dorsal right and left lung areas show slight, but significant relative impedance changes, which run counter to the ventilation. While the impedance otherwise increases during the supply of air, it drops there significantly. The correlations are consequently −80% to −95%. These are not overshoots, whose correlation would equal −100% and thus would also occur at other locations. It is reasonable to suspect that this characteristic is linked with the damaged lung in the dorsal area. Regardless of the exact cause of the anticorrelating characteristic, no ventilation takes place here.

The correlation coefficients c_{Mi} selected as deviation indicators here are compared for all image elements selected through the mask with a threshold cₜₕᵣ. A threshold of cₜₕᵣ=0.5 was set in this example. If the correlation coefficient was below the threshold, the image element in question was assessed being non-ventilated. The percentage of the image elements assessed as being non-ventilated was 6% in the example in FIG. 4.

A display module 23 for a further analysis of the local ventilation of the lung is also shown schematically in FIG. 5; the image elements marked as non-ventilated can be excluded with the EIT device and method according to the present invention in the analysis module 23 from the further analysis of the local ventilation, so that the quality of the further analysis improves.

While specific embodiments of the invention have been shown and described in detail to illustrate the application of the principles of the invention, it will be understood that the invention may be embodied otherwise without departing from such principles.

What is claimed is:

1. An electric impedance tomography device comprising:
a plurality of electrodes, which can be arranged about the chest of a patient;
a control and analyzing unit, which is configured to supply an electrode pair as a feeding electrode pair with an alternating current or with an alternating voltage, to record a voltage signal or current signal as a measured signal from each electrode pair of all other electrode pairs and to let each electrode pair of a plurality of electrode pairs act consecutively as the feeding electrode pair in order to reconstruct, from the measured signals with a reconstruction algorithm, a matrix of image elements, which represents the distribution of the impedance changes in the electrode plane, and to repeatedly record measured signals over time and to reconstruct matrices, wherein the control and analyzing unit is configured to obtain a time series of the impedance change from the sequence of the reconstructed matrices over at least one breath for each image element, to determine a time series of the mean impedance change or a time series of a measured respiration volume and to compare for each image element the corresponding time series of the impedance change with the time series of the mean impedance change or with the time series of the measured respiration volume by a scaled value being calculated for each image element as an indicator of a deviation of the time series of the impedance change of the image element from the time series of the mean impedance change or from the time series of the measured respiration volume and a corresponding image element is assessed and marked as being non-ventilated if an indicator of the deviation meets a preset threshold value criterion.

2. An electric impedance tomography device in accordance with claim 1, wherein the control and analyzing unit calculates a correlation coefficient between the time series of the impedance change and the time series of the mean impedance change or the time series of the measured respiration volume as the indicator of the deviation for each image element and a checking is performed as a threshold value criterion to determine whether the correlation coefficient is below a preset value.

3. An electric impedance tomography device in accordance with claim 1, wherein the control and analyzing unit is configured to calculate for each image element a cross correlation function of the time series of the impedance change and the time series of the mean impedance change or the time series of the measured respiration volume, to determine the maximum of the cross correlation function as an indicator of the deviation, and to perform a check as a threshold value criterion to determine whether the maximum is below a preset value.

4. An electric impedance tomography device in accordance with claim 1, wherein the indicator of the deviation is calculated for each image element by a standardized time series of the impedance change being considered to be a vector and by a standardized time series of the mean impedance change or a standardized time series of the measured respiration volume being considered to be a vector and the norm of the difference of said vectors is calculated as an indicator of the deviation, and the control and analyzing unit checks, as a threshold value criterion, whether the norm of the difference exceeds a preset threshold value, wherein the standardization of the time series is performed by division by the standard deviation thereof or by the median-absolute deviation thereof, by the integral value thereof or by a norm of the vector space being considered.

5. An electric impedance tomography device in accordance with claim 1, wherein the indicator of the deviation is calculated for each image element by considering a standardized time series of the impedance change to be a vector and a standardized time series of the mean impedance change or a standardized time series of the measured respiration volume to be a vector and calculating the scalar product of said vectors as an indicator of the deviation, and that the control and analyzing unit checks, as a threshold value criterion, whether the value of the scalar product is below a preset threshold value, wherein the standardization of the time series is performed by division by the standard deviation thereof or by the median-absolute deviation thereof, by the integral value thereof or by a norm of the vector space being considered.

6. An electric impedance tomography device in accordance with claim 1, wherein the indicator of the deviation is calculated for each image element by considering a standardized time series of the impedance change to be a vector or a standardized time series of the measured respiration volume to be a vector and calculating the norm of the sum of said vectors as an indicator of the deviation, and the control and analyzing unit checks a threshold value criterion to determine whether the norm of the sum is below a preset threshold value, wherein the standardization of the time series is performed by division by the standard deviation thereof or by the median-absolute deviation thereof, by the integral value thereof or by a norm of the vector space being considered.

7. An electric impedance tomography device in accordance with claim 1, wherein the control and analyzing unit is configured to display the image elements assessed as being non-ventilated in an EIT image in a preset shade.

8. An electric impedance tomography device in accordance with claim 1, wherein the control and analyzing unit is configured to display the sum of the areas of the image elements assessed as being non-ventilated or the ratio of the sum to the cross-sectional area of the lung in the EIT image in an EIT image graphically or alphanumerically.

9. An electric impedance tomography device in accordance with claim 1, wherein the control and analyzing unit is configured to perform a reconstruction of the matrices from the measured signals in real time and to determine the indicator of the deviation for each image element in real time and to check the threshold value criterion in real time and to mark image elements assessed as being non-ventilated in a current EIT image.

10. An electric impedance tomography device in accordance with claim 1, wherein the control and analyzing unit is configured to store the measured signals and to perform the reconstruction of the matrices and the formation of the time series of the mean impedance change of the image elements and of the time series of the mean impedance change or of the time series from the stored measured respiration volume in a subsequent analysis at preset times and to determine, for each image element, the indicator of the deviation of the time series of the image element from the time series of the mean impedance change or of the time series of the measured respiration volume and to perform a check for the threshold value criterion and to assess image elements that meet a preset threshold value criterion as being non-ventilated.

11. A method for recording a sequence of EIT images of a cross-sectional plane of the chest of a patient, the method comprising:
providing a control and analyzing unit connected to a plurality of electrodes;
arranging the plurality of electrodes on the circumference of the chest of the patient, wherein one electrode pair as a feeding electrode pair is supplied with an alternating current or an alternating voltage, a voltage signal or current signal is recorded as a measured signal from each electrode pair of all other electrode pairs, and each electrode pair of the plurality of electrode pairs is operated consecutively as the feeding electrode pair; and
with the control and analyzing unit:
reconstructing a matrix of image elements, which represents the distribution of the impedance changes in an electrode plane, from all the measured signals with a reconstruction algorithm,
reconstructing matrices of the impedance change repeatedly over time;
obtaining a time series of the impedance change of the image element from the sequence of the matrices recorded over at least one breath for each image element;
determining a time series of the mean impedance change or a time series of a measured respiration volume is determined at the same times as the time series of the image elements; and
comparing a corresponding time series of the impedance change, for each image element, with the time series of the mean impedance change or with the time series of the measured respiration volume by calculating for each image element a scalar value as an indicator of the deviation of the time series of the impedance change of the image element from the time series of the mean impedance change or of the time series of the measured respiration volume; and
assessing a corresponding image element as being non-ventilated and marking as such if the indicator of the deviation meets a preset threshold value criterion.

12. A method in accordance with claim 11, wherein the matrices are reconstructed in real time, the time series are formed in real time, the indicator of the deviation is determined for each image element in real time, and image elements assessed as being non-ventilated are displayed in a current display of the EIT image.

13. A method in accordance with claim 11, wherein the measured signals are stored and the matrices are reconstructed in a subsequent analysis step, the time series for the impedance changes of the image elements, for the mean impedance change or for the measured respiration volume are formed, and the indicators of the deviations of the image elements are determined at given times and checked for the threshold value criterion, and the image elements that meet a preset threshold value criterion are assessed as being non-ventilated.

14. An electric impedance tomography system comprising:
a plurality of electrodes for being arranged about the chest of a patient;
a control and analyzing unit controlling alternating current or an alternating voltage to a feeding electrode pair of the electrodes, recording a voltage signal or current signal as a measured signal from remaining electrodes and consecutively changing the electrodes that are the feeding electrode pair to reconstruct, from the measured signals with a reconstruction algorithm, a matrix of image elements, which represents the distribution of the impedance changes in the electrode plane, and repeatedly recording measured signals over time to reconstruct matrices, obtaining a time series of the impedance change of the image element from the sequence of the matrices recorded over at least one breath for each image element, determining a time series of the mean impedance change or a time series of a measured respiration volume is determined at the same times as the time series of the image elements, comparing a corresponding time series of the impedance change, for each image element, with the time series of the mean impedance change or with the time series of the measured respiration volume by calculating for each image element a scalar value as an indicator of the deviation of the time series of the impedance change of the image element from the time series of the mean impedance change or of the time series of the measured respiration volume and assessing a corresponding image element as being non-ventilated and marking as such if the indicator of the deviation meets a preset threshold value criterion.

15. An electric impedance tomography system in accordance with claim 14, wherein the control and analyzing unit calculates a correlation coefficient between the time series of the impedance change and the time series of the mean impedance change or the time series of the measured respiration volume as the indicator of the deviation for each image element and the control and analyzing unit performs a checking as a threshold value criterion to determine whether the correlation coefficient is below a preset value.

16. An electric impedance tomography system in accordance with claim 14, wherein the control and analyzing unit calculates, for each image element, a cross correlation function of the time series of the impedance change and the time series of the mean impedance change or the time series of the measured respiration volume, to determine the maximum of the cross correlation function as an indicator of the deviation, and performs a check as a threshold value criterion to determine whether the maximum is below a preset value.

17. An electric impedance tomography system in accordance with claim 14, wherein the indicator of the deviation is calculated for each image element by a standardized time series of the impedance change being considered to be a vector and by a standardized time series of the mean impedance change or a standardized time series of the measured respiration volume being considered to be a vector and the norm of the difference of said vectors is calculated as an indicator of the deviation, and the control and analyzing unit checks, as a threshold value criterion, whether the norm of the difference exceeds a preset threshold value, wherein the standardization of the time series is performed by division by the standard deviation thereof or by the median-absolute deviation thereof, by the integral value thereof or by a norm of the vector space being considered.

18. An electric impedance tomography system in accordance with claim 14, wherein the indicator of the deviation is calculated for each image element by considering a standardized time series of the impedance change to be a vector and a standardized time series of the mean impedance change or a standardized time series of the measured respiration volume to be a vector and calculating the scalar product of said vectors as an indicator of the deviation, and that the control and analyzing unit checks, as a threshold value criterion, whether the value of the scalar product is below a preset threshold value, wherein the standardization of the time series is performed by division by the standard deviation thereof or by the median-absolute deviation thereof, by the integral value thereof or by a norm of the vector space being considered.

19. An electric impedance tomography system in accordance with claim 14, wherein the indicator of the deviation is calculated for each image element by considering a standardized time series of the impedance change to be a vector or a standardized time series of the measured respiration volume to be a vector and calculating the norm of the sum of said vectors as an indicator of the deviation, and the control and analyzing unit checks a threshold value criterion to determine whether the norm of the sum is below a preset threshold value, wherein the standardization of the time series is performed by division by the standard deviation thereof or by the median-absolute deviation thereof, by the integral value thereof or by a norm of the vector space being considered.

20. An electric impedance tomography system in accordance with claim 14, wherein the marking includes at least one of:
displaying the image elements assessed as being non-ventilated in an EIT image in a preset shade; and
displaying a sum of the areas of the image elements assessed as being non-ventilated or the ratio of the sum to the cross-sectional area of the lung in the EIT image in an EIT image graphically or alphanumerically.
